(19) United States
(12) Patent Application Publication
(10) Pub. No.: US 2026/0232696 A1
(43) Pub. Date: Aug. 13, 2026

(54) IDENTIFICATION OF A KEY GENE FOR REVERSION OF CANCER CELLS INTO NORMAL CELLS AND DRUG REPURPOSING

(71) Applicant: biorevert Inc., Daejeon (KR)

(72) Inventors: Kwang Hyun CHO, Daejeon (KR); Jeong Ryeol GONG, Daejeon (KR); Kyeong Hwan HAN, Daejeon (KR)

(73) Assignee: biorevert Inc., Daejeon (KR)

(21) Appl. No.: 19/504,213

(22) PCT Filed: Jul. 2, 2025
(86) PCT No.: PCT/KR2025/009402

§ 371 Date: Jan. 21, 2026

(30) Foreign Application Priority Data
Jul. 12, 2024 (KR) 10-2024-0092183
Jul. 1, 2025 (KR) 10-2025-0087787

(51) Int. Cl. A61K 31/585; A61K 31/167; A61P 35/00; G01N 33/50

(52) U.S. Cl. CPC A61K 31/585; A61K 31/167; A61P 35/00; G01N 33/5011

(57) ABSTRACT

A pharmaceutical composition that includes spironolactone or BPK-21, or a pharmaceutically acceptable salt thereof, as an active ingredient, wherein the composition specifically inhibits or degrades ERCC3 (ERCC Excision Repair 3, TFIIH core complex helicase subunit), thereby suppressing proliferation of cancer cells and simultaneously inducing normal differentiation. A method for screening a drug that reverts cancer cells into normal-like cells (or normal-functioning cells) by specifically inhibiting or degrading ERCC3 that includes: (a) treating cancer cells with a candidate substance in vitro; (b) measuring the expression level of ERCC3 in the treated cancer cells; (c) selecting a candidate substance that exhibits a lower expression level of ERCC3 than that of a control group; and (d) determining, among the candidate substances, one that reduces expression of at least one of KRAS, HMGA1, and G3BP1 or increases expression of BMP2, as a drug that reverts cancer cells into normal-like cells (or normal-functioning cells).

### TECHNICAL FIELD

The present invention relates to a pharmaceutical composition for preventing or treating cancer, which reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation.

Moreover, the present invention relates to a method for screening a drug that reverts cancer cells into normal-like cells (or normal-functioning cells) by specifically inhibiting or degrading ERCC3, thereby suppressing the proliferation of cancer cells and simultaneously inducing normal differentiation.

### BACKGROUND ART

Anticancer agents are generally classified into cytotoxic chemotherapeutic agents, targeted anticancer agents, and immunotherapeutic anticancer agents. Cytotoxic chemotherapeutic agents kill cancer cells by exploiting the characteristic of cancer cells proliferating faster than normal cells, and, in general, combination chemotherapy, which generally employs drugs having different mechanisms of action, is used to enhance therapeutic efficacy and inhibit cross-resistance. Cytotoxic chemotherapeutic agents also affect normal cells, particularly blood cells, hair follicle cells, mucosal cells, and reproductive cells, which proliferate at a rate comparable to that of cancer (tumor) cells, resulting in characteristic side effects such as bone marrow suppression (decrease in leukocytes, erythrocytes, and platelets), alopecia, stomatitis, diarrhea, and infertility. Targeted anticancer agents are drugs designed to target overexpressed proteins that induce cancer-associated characteristics. By blocking specific proteins, they either inhibit the formation of blood vessels necessary for cancer growth and metastasis or delay cancer growth. Compared with cytotoxic chemotherapeutic agents, targeted anticancer agents exert less influence on normal cells and thus cause fewer side effects. However, cancer cells can rapidly adapt to targeted anticancer agents and acquire resistance. Recently emerging immunotherapeutic anticancer agents act by enhancing immune responses through blocking the immunosuppressive effects of cancer cells. However, in the case of immunotherapeutic anticancer agents, for example, the response rate is low, and therefore they can be applied only to a small number of patients. In addition, there is a risk of recurrence during long-term follow-up, and fatal side effects such as myocarditis have also been reported.

Conventional cancer differentiation therapy strategies indirectly inhibit cancer cell properties by inducing the differentiation of cancer cells, resulting in limited effects. Moreover, such strategies have been applied only to certain cancer types, such as neuroblastoma, thyroid cancer, and some hematologic malignancies (e.g., acute promyelocytic leukemia and certain subtypes of acute myeloid leukemia). The primary reason for such limited effects is that the differentiation state of cancer cells and the cancer cell properties are often regulated independently of each other, so that simple induction of differentiation alone cannot effectively inhibit the inherent unlimited proliferative capacity of cancer cells. Meanwhile, in some cases of colorectal cancer, even in stage IV cancer patients with distant metastasis, well-differentiated primary cancers have been observed.

Based on the observation that, in the process of carcinogenesis, normal-like cells (or normal-functioning cells) undergo carcinogenic transformation through two key axes, namely, acquisition of stemness (dedifferentiation potential) and acquisition of cancer cell properties, there is a need for a strategy to “revert cancer cells into normal-like cells (or normal-functioning cells)” by simultaneously inhibiting the dedifferentiation potential of cancer cells and the acquisition of cancer cell properties, thereby reversing the process of carcinogenesis.

The induction of normal differentiation of cancer cells and the reduction of cancer cell properties can be considered as the two most important axes in cancer therapy. The inventors of the present invention have developed a framework capable of measuring the differentiation of cancer cells by knocking out all possible genes in a computer model of a gene network, and have identified, based on this framework, a novel target, ERCC3 (ERCC Excision Repair 3, TFIIH core complex helicase subunit), also referred to as XPB (Xeroderma Pigmentosum group B), which can induce the differentiation of cancer cells and simultaneously reduce cancer cell properties.

Furthermore, the inventors have demonstrated that inhibition of ERCC3 exerts little or no effect on normal cells, while suppressing both the dedifferentiation potential and the cancer cell properties of cancer cells, thereby making it possible to achieve cancer treatment, alleviation, or prevention without side effects through the reversion of cancer cells into normal-like cells (or normal-functioning cells).

In addition, the inventors have demonstrated that small-molecule compounds, such as spironolactone, which have conventionally been used as anti-aldosterone agents and potassium-sparing diuretics for the treatment of hypertension, edema, heart failure, ascites caused by cirrhosis, primary aldosteronism, polycystic ovary syndrome, androgen-excess symptoms (such as acne and hirsutism), and hypokalemia, exhibit remarkably superior effects in promoting the reversion of cancer cells into normal-like cells (or normal-functioning cells) through inhibition or degradation of ERCC3.

Additionally, the inventors have confirmed that spironolactone suppresses the proliferation (growth) of cancer cells and induces their differentiation in solid cancers such as colorectal cancer, breast cancer, lung cancer, and skin cancer (particularly melanoma), thereby demonstrating that a reversion therapy strategy based on the inhibition or degradation of ERCC3 can be applied at the pan-cancer level.

### DISCLOSURE

### Technical Problem

Accordingly, an object of the present invention is to provide a pharmaceutical composition for preventing or treating cancer, which reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation. That is, the object of the present invention is to enable cancer treatment, alleviation, or prevention without side effects through reversion of cancer cells into normal-like cells (or normal-functioning cells), by simultaneously inducing the differentiation of cancer cells and suppressing the cancer cell properties, while exerting little or no effect on normal-like cells (or normal-functioning cells).

Another object of the present invention is to provide a method for screening a drug that reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation.

### Technical Solution

The present invention provides a pharmaceutical composition for preventing or treating cancer, comprising spironolactone represented by Chemical Formula 1 below or a pharmaceutically acceptable salt thereof, or BPK-21 represented by Chemical Formula 2 below or a pharmaceutically acceptable salt thereof, as an active ingredient, wherein the composition reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation:

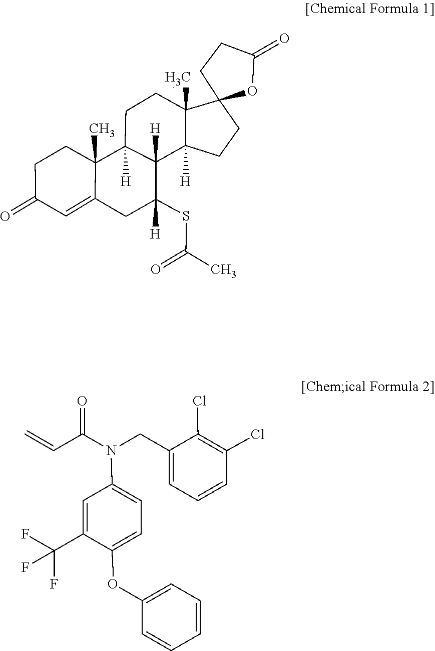

In the composition for preventing or treating cancer according to the present invention, spironolactone (Aldactone®) or a pharmaceutically acceptable salt thereof, or BPK-21 or a pharmaceutically acceptable salt thereof, as an active ingredient, reduces the expression of at least one gene selected from the group consisting of KRAS (Kirsten rat sarcoma viral oncogene homolog), HMGA1 (High-Mobility Group AT-Hook 1), and G3BP1 (GTPase Activating Protein (SH3 Domain) Binding Protein 1). ERCC3 directly induces the transcription of KRAS, thereby promoting the proliferation of cancer cells. Furthermore, ERCC3 increases the expression of HMGA1 and G3BP1, thereby activating the WNT signaling pathway, which in turn enhances the stem cell properties of cancer cells. Specific binding of ERCC3 to the promoter regions of KRAS, HMGA1, and G3BP1 has been confirmed, and the inhibition or degradation of ERCC3 has been shown to significantly reduce the expression of these genes. Accordingly, ERCC3 is considered to directly participate in the transcriptional activity of these genes and contribute to their transcriptional activation. The genes bound by ERCC3 (KRAS, HMGA1, and G3BP1) have functions that contribute to the proliferation and development of cancer, and particularly include features associated with malignant progression of cancer, such as epithelial-mesenchymal transition (EMT). KRAS is closely associated with the proliferation of colorectal cancer cells, and HMGA1 and G3BP1 are closely associated with the proliferation of cancer cells and the activation of stemness through the activation of the WNT signaling pathway. Therefore, spironolactone (Aldactone®) or a pharmaceutically acceptable salt thereof, or BPK-21 or a pharmaceutically acceptable salt thereof, suppresses the proliferation of cancer cells and inhibits stemness.

In the composition for preventing or treating cancer according to the present invention, spironolactone (Aldactone®) or a pharmaceutically acceptable salt thereof, or BPK-21 or a pharmaceutically acceptable salt thereof, as an active ingredient, increases the expression of the BMP2 (Bone Morphogenetic Protein 2) gene. Spironolactone (Aldactone®) or a pharmaceutically acceptable salt thereof, or BPK-21 or a pharmaceutically acceptable salt thereof, specifically inhibits or degrades ERCC3, and when the expression of ERCC3 is inhibited, the expression of BMP2 is significantly increased, ultimately inducing cancer cells to undergo normal differentiation into normal-like cells (or normal-functioning cells).

In the composition for preventing or treating cancer according to the present invention, spironolactone (Aldactone®) or a pharmaceutically acceptable salt thereof, or BPK-21 or a pharmaceutically acceptable salt thereof, as an active ingredient, reduces the abnormal overexpression of cancer-related genes such as MYC and cell proliferation-related genes such as MK167 and PCNA in cancer cells. Inhibition or degradation of ERCC3 suppresses the KRAS (associated with the MAPK pathway) and WNT signaling pathways, thereby reducing the abnormal overexpression of cancer-related genes such as MYC and cell proliferation-related genes such as MKI67 and PCNA in cancer cells.

In the composition for preventing or treating cancer according to the present invention, spironolactone (Aldactone®) or a pharmaceutically acceptable salt thereof, or BPK-21 or a pharmaceutically acceptable salt thereof, as an active ingredient, specifically increases the expression of KRT20 (Keratin 20), or KLF4 (KLF Transcription Factor 4), or both KRT20 and KLF4, which are differentiation markers of normal colon cells. The increase in the expression of KRT20, or KLF4, or both KRT20 and KLF4 indicates that colorectal cancer cells are induced to revert into normal colon-like cells (or normal colon-functioning cells).

Moreover, in the composition for preventing or treating cancer according to the present invention, spironolactone (Aldactone®) or a pharmaceutically acceptable salt thereof, or BPK-21 or a pharmaceutically acceptable salt thereof, as an active ingredient, specifically increases the expression of GATA3 (GATA Binding Protein 3), which is a differentiation marker of normal breast cells. The increase in the expression of GATA3 indicates that breast cancer cells are induced to revert into normal breast-like cells (or normal breast-functioning cells).

Furthermore, in the composition for preventing or treating cancer according to the present invention, spironolactone (Aldactone®) or a pharmaceutically acceptable salt thereof, or BPK-21 or a pharmaceutically acceptable salt thereof, as an active ingredient, specifically increases the expression ofNKX2-1 (NK2 homeobox 1), or SCGB1A1 (secretoglobin family TA member 1), or both NKX2-1 and SCGBTAT, which are differentiation markers of normal lung cells. The increase in the expression ofNKX2-1, or SCGBlA1, or both NKX2-1 and SCGB1A1 indicates that lung cancer cells are induced to revert into normal lung-like cells (or normal lung-functioning cells).

In addition, in the composition for preventing or treating cancer according to the present invention, spironolactone (Aldactone®) or a pharmaceutically acceptable salt thereof, or BPK-21 or a pharmaceutically acceptable salt thereof, as an active ingredient, specifically increases the expression of KRT1 (Keratin 1), which is a differentiation marker of normal skin cells. The increase in the expression of KRT1 indicates that melanoma cells are induced to revert into normal skin-like cells (or normal skin-functioning cells).

The composition for preventing or treating cancer according to the present invention exhibits a pan-cancer level reversion effect, which reduces cancer cell properties in various cancer types, including colorectal cancer, breast cancer, lung cancer, and skin cancer (e.g., melanoma), while inducing normal differentiation into normal-like cells (or normal-functioning cells).

Additionally, the present invention provides a method for screening a drug that reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation, the method comprising: (a) treating cancer cells with a candidate substance in vitro; (b) measuring the expression level of ERCC3 in the cancer cells treated with the candidate substance; (c) selecting a candidate substance that exhibits a lower expression level of ERCC3 than that of a control group not treated with the candidate substance; and (d) determining, among the candidate substances selected in step (c), a candidate substance that reduces the expression of at least one gene selected from the group consisting of KRAS, HMGA1, and G3BP1 or increases the expression of the BMP2 gene, as a drug that reverts cancer cells into normal-like cells (or normal-functioning cells).

In the method for screening a drug according to the present invention, which reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation, the drug that reverts cancer cells into normal-like cells (or normal-functioning cells) induces a decrease in the expression of cancer-related genes such as MYC and also reduces the overexpression of cell proliferation-related genes such as MK167 and PCNA in cancer cells.

In the method for screening a drug according to the present invention, which reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation, the drug that reverts cancer cells into normal-like cells (or normal-functioning cells) specifically increases the expression of KRT20 (Keratin 20), or KLF4 (KLF Transcription Factor 4), or both KRT20 and KLF4, which are differentiation markers of normal colon cells. The increase in the expression of KRT20, or KLF4, or both KRT20 and KLF4 indicates that colorectal cancer cells are induced to revert into normal colon-like cells (or normal colon-functioning cells).

In the method for screening a drug according to the present invention, which reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation, the drug that reverts cancer cells into normal-like cells (or normal-functioning cells) specifically increases the expression of GATA3 (GATA Binding Protein 3), which is a differentiation marker of normal breast cells. The increase in the expression of GATA3 indicates that breast cancer cells are induced to revert into normal breast-like cells (or normal breast-functioning cells).

In the method for screening a drug according to the present invention, which reverts cancer cells into normal-like cells (or normal-functioning cells) by specifically inhibiting ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation, the drug that reverts cancer cells into normal-like cells (or normal-functioning cells) specifically increases the expression of NKX2-1 (NK2 homeobox 1), or SCGB1A1 (secretoglobin family TA member 1), or both NKX2-1 and SCGBlA1, which are differentiation markers of normal lung cells. The increase in the expression of NKX2-1, or SCGBlA1, or both NKX2-1 and SCGB1A1 indicates that lung cancer cells are induced to revert into normal lung-like cells (or normal lung-functioning cells).

In the method for screening a drug according to the present invention, which reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation, the drug that reverts cancer cells into normal-like cells (or normal-functioning cells) specifically increases the expression of KRT1 (Keratin 1), which is a differentiation marker of normal skin cells. The increase in the expression of KRT1 indicates that melanoma cells are induced to revert into normal skin-like cells (or normal skin-functioning cells).

ERCC3 is a key target gene that reverts cancer cells into normal-like cells (or normal-functioning cells), and specific inhibition or degradation of ERCC3 can be utilized as a cancer reversion therapy strategy that suppresses cancer cell properties while promoting normal differentiation.

### Effects of the Invention

When the expression of the ERCC3 gene is suppressed in colorectal cancer cell lines and normal colon cell lines, the proliferation is inhibited in the colorectal cancer cell lines and the gene expression similar to that of normally differentiated colon epithelial cell lines is observed, whereas proliferation (growth) is not inhibited in the normal colon cell lines. Therefore, inhibition or degradation of ERCC3 can induce differentiation and suppress cancer cell properties specifically in colorectal cancer cells without affecting normal colon cell lines. Inhibition or degradation of ERCC3 has been shown to suppress the proliferation of cancer cells and induce normal differentiation not only in colorectal cancer but also in various solid cancers, including breast cancer, lung cancer, and skin cancer (particularly melanoma). These findings demonstrate that a reversion therapy strategy based on the inhibition or degradation of ERCC3 can be applied at the pan-cancer level.

### BRIEF DESCRIPTION OF THE DRAWINGS

FIGS. 1A to 1K summarize the overall analytical process and key results for the identification and functional verification of ERCC3, a drug target gene for inducing the reversion of colorectal cancer cells, based on in silico simulation using a gene regulatory network.

FIG. 1A illustrates an overview of the experimental design. The left panel shows the process of collecting tissue samples from tumors and adjacent normal tissues of colorectal cancer patients and generating colorectal cancer organoids and normal colon organoids from the collected samples. The central panel shows the process of generating single-cell RNA-seq transcriptome data and constructing a gene regulatory network that reflects cell states based on the data. The right panel illustrates the analytical workflow of performing in silico gene knockout simulations for all genes to identify candidate drug target genes capable of inducing the redifferentiation of cancer cells.

FIG. 1B illustrates the results of a UMAP (Uniform Manifold Approximation and Projection) analysis, which is a low-dimensional mapping of single-cell transcriptome data.

FIG. 1C illustrates a dot plot representing the expression levels of representative marker genes and the proportions of expressing cells in each cell cluster.

FIG. 1D illustrates a comparison of the proportions of each cell type between normal colon organoids and colorectal cancer organoids.

FIG. 1E visualizes the separation of a stem cell cluster into two sub-clusters through clustering analysis based on the results of low-dimensional mapping using UMAP (Uniform Manifold Approximation and Projection).

FIG. 1F illustrates boxplots comparing the signature scores of MYC target genes, cancer signatures, and cell proliferation-related signatures between stem cell sub-clusters.

FIG. 1G illustrates the results of a transcriptional age index (TAI) analysis.

FIG. 1H illustrates a comparison of the signature scores of differentially regulated (DR) genes regulated by ERCC3 between Cluster 1 and differentiated enterocytes.

FIG. 1I illustrates the results of gene ontology (GO) analysis of DR genes regulated by ERCC3.

FIG. 1J illustrates a violin plot comparing the expression levels of the ERCC3 gene.

FIG. 1K illustrates a Kaplan-Meier survival curve analyzing the survival rates according to the expression levels of the ERCC3 gene, based on colorectal cancer patient data from TCGA (The Cancer Genome Atlas).

FIGS. 2A to 2F illustrate the analysis of the gene regulatory function of ERCC3 and its role in promoting carcinogenesis in colorectal cancer, and shows the results of evaluating the effect of ERCC3 on the regulation of gene expression during carcinogenesis.

FIG. 2A illustrates the experimental design of the ChIP-seq (Chromatin Immunoprecipitation sequencing) analysis to identify the DNA-binding sites of ERCC3 in the colorectal cancer cell line HT29.

FIG. 2B illustrates the results of ChIP-seq analysis, showing that more than 90% of the total binding sites of ERCC3 are located in distal intergenic and intron regions, and that approximately 4.4% of the total binding sites are located in promoter regions of genes.

FIG. 2C illustrates a comparison to determine whether ERCC3 actually binds to promoter regions in the genome, by examining whether the signals of H3K4me3 (an active promoter marker) and RNA polymerase II appear around ERCC3 ChIP-seq peaks.

FIG. 2D illustrates the results of identifying the gene promoters to which ERCC3 primarily binds, showing that specific binding peaks of ERCC3 are present in the promoter regions of KRAS, HMGA1, and G3BPT.

FIG. 2E illustrates the results of gene ontology (GO) analysis performed to evaluate the common functions of ERCC3 target genes.

FIG. 2F summarizes FIGS. 2A to 2E above, illustrating that ERCC3 promotes the proliferation of cancer cells through the expression of KRAS, and induces activation of the WNT signaling pathway and an increase in sternness through the expression of HMGA1 and G3BP1, thereby enhancing the cancer cell properties (proliferation and dedifferentiation features) of colorectal cancer.

FIGS. 3A to 3G illustrate the analysis of the mechanism by which inhibition of ERCC3 expression suppresses the KRAS-MAPK and WNT signaling pathways, thereby inhibiting colorectal cancer proliferation.

FIG. 3A illustrates the results of gene set enrichment analysis (GSEA) performed to confirm that the unlimited proliferative capacity of colorectal cancer is closely associated with activation of the KRAS (linked to the MAPK pathway) and WNT pathways, and to evaluate whether the reduction of ERCC3 expression leads to the inhibition of these pathways.

FIG. 3B illustrates the results of western blot analysis performed in the HT29 cell line to verify the experimental results of (A) above at the protein level.

FIG. 3C illustrates the results of gene ontology (GO) analysis performed to evaluate how the activation of KRAS and WNT pathways actually affects downstream proliferation-related genes.

FIG. 3D illustrates the results of evaluating the effect of ERCC3 inhibition on cell proliferation in three colorectal cancer cell lines (HT29, HCT116, and CACO2).

FIG. 3E illustrates that ERCC3, a reversion target of colorectal cancer cells, has little or no effect on the proliferation of normal colon cells.

FIG. 3F and FIG. 3g illustrate the results of evaluating the effect of ERCC3 inhibition after transplantation of three colorectal cancer cell lines (HT29, HCT116, and CACO2) into mice.

FIGS. 4A to 4G illustrate the mechanism by which inhibition of ERCC3 induces the expression of BMP2 and promotes the differentiation of colorectal cancer cells into normal-like (or normal-functioning) colon cells.

FIG. 4A illustrates that inhibition of ERCC3 in the colorectal cancer cell line HT29 decreases cancer cell characteristics along the X-axis while increasing differentiation characteristics toward normal epithelial cells along the Y-axis.

FIG. 4B illustrates the results of gene expression analysis performed to determine whether the expression of genes associated with normal colon functions is restored when differentiation is induced in colorectal cancer cells.

FIG. 4C illustrates that inhibition of ERCC3 increases the expression of key genes essential for maintaining the polarity and barrier functions of colon epithelial cells, including genes related to the brush border, tight junction, and apical junction complex, which are representative structural features of normal colon tissue.

FIG. 4D illustrates that inhibition of ERCC3 in two colorectal cancer cell lines (HT29 and CACO2) increases the expression of both KRT20 and KLF4, which are differentiation markers of colon epithelial cells.

FIG. 4E and FIG. 4F illustrate that the differentiation of colorectal cancer cells into normal colon epithelial cells is closely associated with increased BMP2 expression.

FIG. 4G illustrates the mechanism by which inhibition of ERCC3 suppresses the WNT signaling pathway, thereby inducing BMP2 activation and promoting the differentiation of colorectal cancer cells into normal colon epithelial cells.

FIGS. 5A to 5K illustrate that spironolactone, a drug that specifically degrades ERCC3, suppresses the proliferation of colorectal cancer cells and promotes differentiation, thereby inducing the reversion of colorectal cancer.

FIG. 5A illustrates that the treatment of five colorectal cancer cell lines corresponding to consensus molecular subtypes (CMSs) 1-4 (CMS1: SW48, CMS2: NCI-H508, CMS3: HT29, and CMS4: HCT116 and CACO2) with spironolactone suppresses proliferation and induces differentiation through ERCC3-specific degradation.

FIG. 5B illustrates that the treatment of five colorectal cancer cell lines corresponding to consensus molecular subtypes (CMSs) 1-4 (CMS1: SW48, CMS2: NCI-H508, CMS3: HT29, and CMS4: HCT116 and CACO2) with spironolactone decreases cell proliferation in a dose-dependent manner.

FIG. 5C illustrates that the treatment of five colorectal cancer cell lines corresponding to consensus molecular subtypes (CMSs) 1-4 (CMS1: SW48, CMS2: NCI-H508, CMS3: HT29, and CMS4: HCT116 and CACO2) with spironolactone increases the expression of KRT20 and KLF4, which are markers of normal colon cell differentiation.

FIG. 5D illustrates that the treatment of the normal colon cell line (NCM460) with spironolactone shows no change in cell proliferation.

FIG. 5E illustrates that the treatment of the normal colon cell line (NCM460) with spironolactone promotes the degradation of ERCC3.

FIG. 5F illustrates the calculated IC₅₀ values of spironolactone in five colorectal cancer cell lines corresponding to consensus molecular subtypes (CMSs) 1-4 (CMST: SW48, CMS2: NCI-H508, CMS3: HT29, and CMS4: HCT116 and CACO2) and the normal colon cell line (NCM460). This demonstrates that colorectal cancer cell lines are sensitive to spironolactone treatment, whereas the normal colon cell line is insensitive to spironolactone treatment.

FIG. 5G illustrates the results of analyzing the extent of cell death in the colorectal cancer cell line (HT29) and the normal colon cell line (NCM460) after treatment with either ERCC3 inhibition or spironolactone. This demonstrates that the reduction of ERCC3, either by direct inhibition of ERCC3 gene expression or by treatment with spironolactone at concentrations that induce reversion therapeutic effects, does not induce apoptosis in either cancer cells or normal colon cells.

FIG. 5H illustrates that the oral administration of spironolactone to mice transplanted with colorectal cancer in the skin significantly reduces tumor size and proliferation rate.

FIG. 5I illustrates that the intraperitoneal administration of spironolactone to mice transplanted with colorectal cancer in the skin demonstrates a tumor-suppressive effect.

FIG. 5J illustrates that both oral and intraperitoneal administration of spironolactone to mice transplanted with colorectal cancer into the intestine demonstrates a tumor-suppressive effect.

FIG. 5K illustrates histological images of the liver and kidney after oral administration of spironolactone to mice. This demonstrates that drug concentrations of spironolactone capable of inducing reversion therapeutic effects do not cause toxicity in the liver and kidney of mice.

FIGS. 6A and 6B illustrate the induction of colorectal cancer reversion by BPK-21, a drug that specifically inhibits ERCC3.

FIG. 6A illustrates that the treatment of five colorectal cancer cell lines CMS1: SW48, CMS2: NCI-H508, CMS3: HT29, and CMS4: HCT116 and CACO2) with BPK-21 significantly decreases the proliferation of colorectal cancer cells.

FIG. 6B illustrates that the treatment of three colorectal cancer cell lines (HT29, HCT116, and CACO2) with BPK-21 increases the expression of KRT20 and KLF4, which are markers of normal colon cell differentiation.

FIGS. 7A and 7B illustrate that spironolactone, a drug that specifically degrades ERCC3, suppresses the proliferation of cancer cells and induces differentiation in breast cancer, lung cancer, and skin cancer (e.g., melanoma).

FIG. 7A illustrates that the treatment of the breast cancer cell line MCF7, the lung cancer cell line A549, and the skin cancer cell line A375 with spironolactone significantly decreases the proliferation of cancer cells.

FIG. 7B illustrates that the treatment of the breast cancer cell line MCF7, the lung cancer cell line A549, and the skin cancer cell line A375 with spironolactone increases the expression of BMP2, a factor that induces cell differentiation, as well as cell differentiation markers including: GATA3, a differentiation marker of normal breast luminal cells; NKX2-1 and SCGBTAT, differentiation markers of normal alveolar type I (AT1) lung cells; and KRT1, a marker of normal skin keratinocytes.

### MODE FOR CARRYING OUT THE INVENTION

The following examples are provided to illustrate preferred embodiments of the present invention. The following examples should not be construed as limiting the scope of the present invention. Unless otherwise defined, all technical and scientific terms used herein have the same meaning as commonly understood by those having ordinary skill in the art to which the present invention pertains.

ERCC3 (ERCC Excision Repair 3, TFIIH core complex helicase subunit), also referred to as XPB (Xeroderma Pigmentosum group B), is a gene that encodes the TFIIH core complex helicase subunit, which is involved in DNA repair and transcription. ERCC3 (ERCC Excision Repair 3, TFIIH core complex helicase subunit, also referred to as XPB, Xeroderma Pigmentosum group B) protein functions as a helicase that unwinds the DNA double helix during DNA replication or damage repair.

Normal cells, which are the basic units that make up all tissues and organs of the human body, divide and grow according to a defined cell cycle, and when damaged, undergo apoptosis, a programmed cell death process, to maintain a balanced cell population. The main characteristics of normal cells include proper regulation of the cell cycle, the ability to differentiate, contact inhibition, a finite lifespan, and a normal morphology (under a microscope, they exhibit a regular shape and size and a consistent ratio of nucleus to cytoplasm).

Cancer cells are abnormally transformed cells that arise from mutations in the genes of normal cells. Cancer cells lose their normal cellular regulatory functions and acquire malignant characteristics that enable them to proliferate indefinitely, invade surrounding tissues, and metastasize to other organs. Compared with normal cells, the main characteristics of cancer cells include unlimited proliferative, abnormal differentiation, loss of contact inhibition, evasion of apoptosis, invasion and metastasis, and abnormal morphology (under a microscope, they exhibit enlarged and irregular nuclei, abnormal cytoplasm, and an increased nucleus-to-cytoplasm ratio).

The term “normal-like cells” or “normal-functioning cells” is used in various contexts in the fields of medicine and biology; however, in the present invention, it refers to cells having transcriptome expression, functions, and morphology similar to those of normal cells.

### Example

### Identification of ERCC3 as Target Gene for Colorectal Cancer Reversion

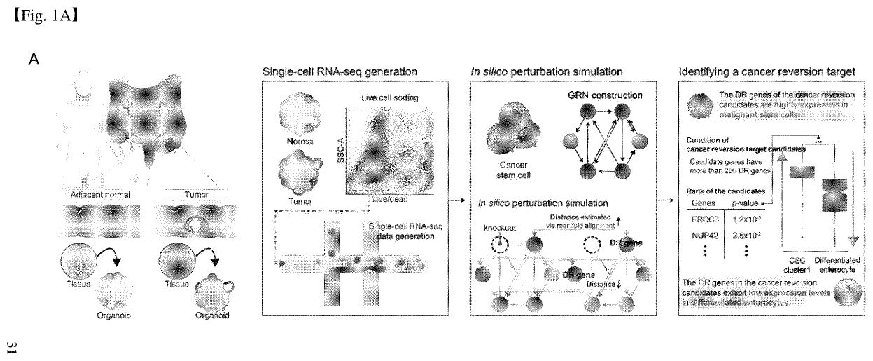

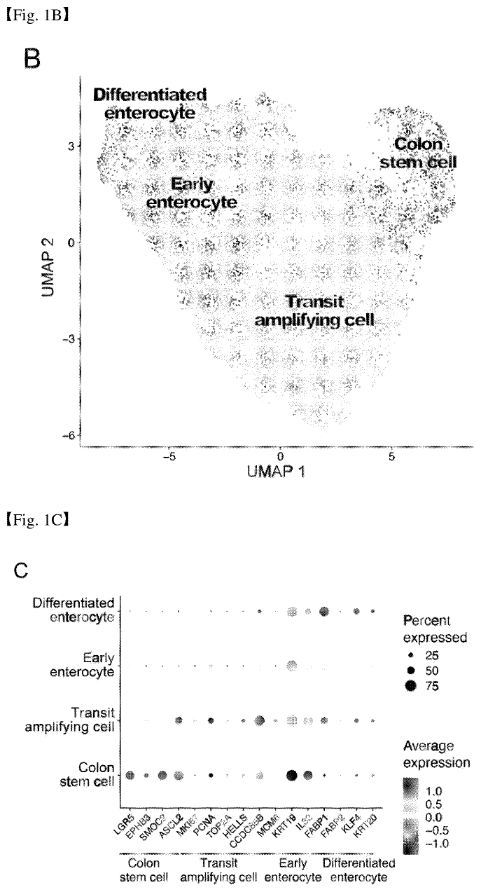

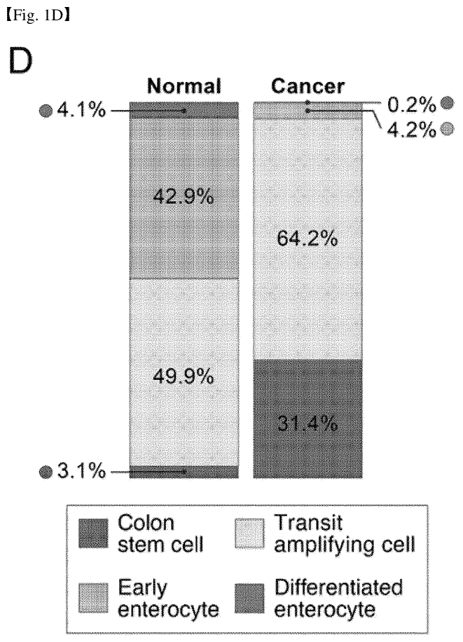

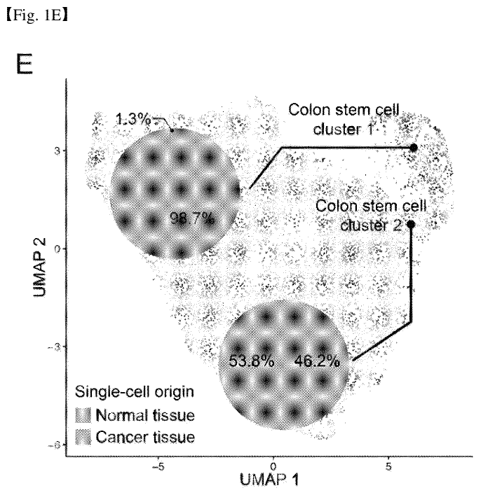

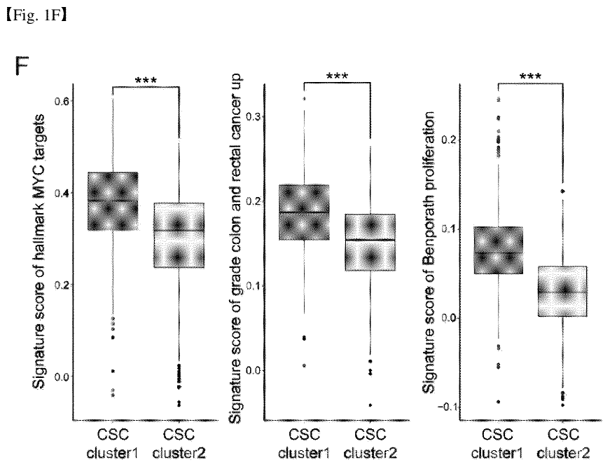

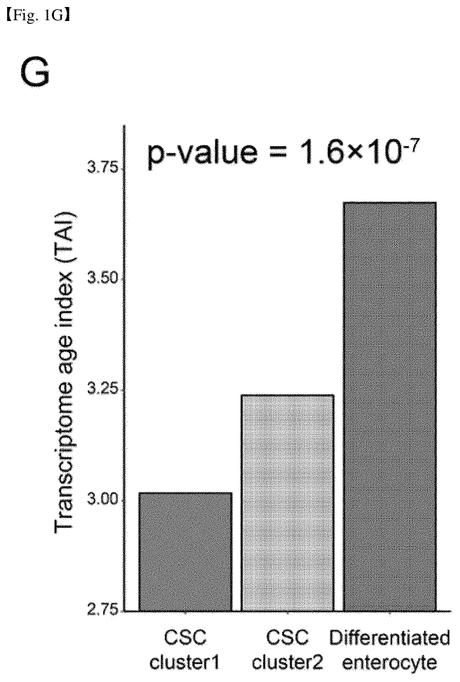

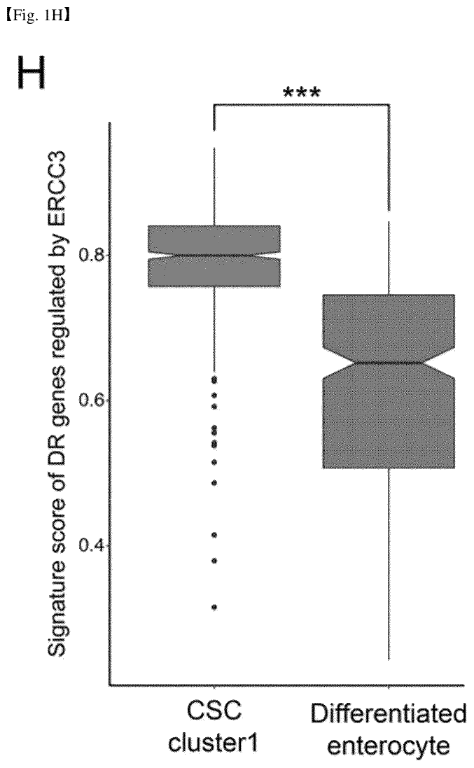

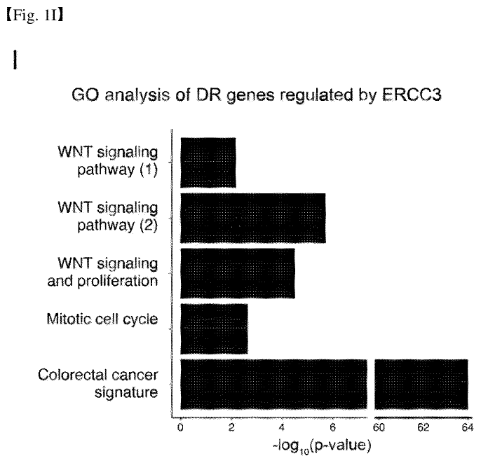

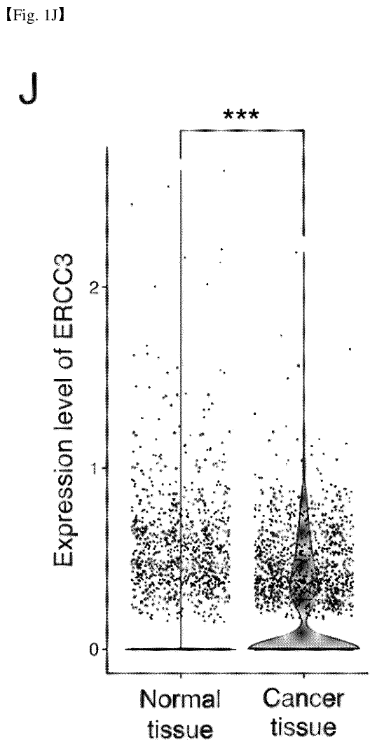

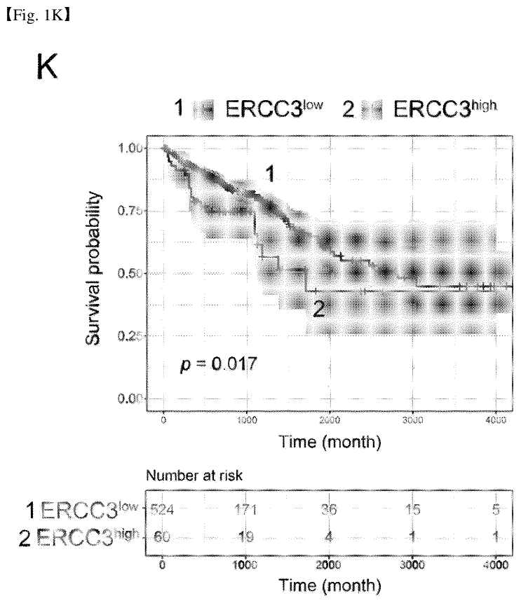

From colorectal cancer organoid-normal colon organoid pairs generated from tumors and adjacent normal tissues of colorectal cancer patients, the two organoids were dissociated into single cells, and single-cell transcriptome data were obtained to analyze cell distribution (see the left panel of FIG. 1(A)). Viable cells were selected to generate single-cell RNA-seq transcriptome data, and based on these data, a gene regulatory network reflecting cell states was constructed (see the central panel of FIG. 1(A)). Based on single-cell transcriptome data, a gene regulatory network was constructed, and an in silico gene knockout simulation was performed for all genes to identify candidate drug target genes capable of inducing the differentiation of cancer cells. Through this analysis, candidate drug target genes that could induce normal differentiation of cancer cells and contribute to cancer cell inhibition were identified. The selection of candidate genes was performed based on a gene set exhibiting relatively low expression in normal organoids and high expression in cancer organoids. This strategy prioritized genes that, when inhibited, are more likely to contribute to the restoration of normal cellular function (see the right panel of FIG. 1(A)). It was found that differentially regulated (DR) genes of candidate drugs for cancer reversion are highly expressed in malignant stem cells and expressed at low levels in differentiated normal enterocytes. From this analysis, ERCC3 (ERCC Excision Repair 3, TFIIH core complex helicase subunit), also referred to as XPB (Xeroderma Pigmentosum group B), was identified.

A low-dimensional mapping analysis of single-cell transcriptomes was performed using UMAP (Uniform Manifold Approximation and Projection). As a result of the analysis, various cell clusters present in colorectal cancer organoids and normal colon organoids were identified. The identified cell clusters consisted of colon stem cells, transit-amplifying cells, early enterocytes, and differentiated enterocytes. FIG. 1(C) illustrates the expression levels of representative marker genes and the proportions of expressing cells in each cell cluster. It was confirmed that KRT20 and KLF4 are representative marker genes of fully differentiated enterocytes. When comparing the proportions of each cell type in normal colon organoids and colorectal cancer organoids, the proportions of stem cells and transit-amplifying cells were found to be increased in colorectal cancer organoids (see FIG. 1(D)).

Clustering analysis based on the results of the low-dimensional mapping using UMAP (Uniform Manifold Approximation and Projection) visualized the separation of the stem cell cluster into two sub-clusters (see FIG. 1(E)). Cluster 1 is manly composed of colorectal cancer cells, and this cell cluster was classified as colorectal cancer stem cells. When comparing the MYC target genes, cancer signatures, and cell proliferation-related signature scores between the stem cell sub-clusters, it was found that cancer-related signatures were more highly activated in Cluster 1, which transformed into cancer stem cells (see FIG. 1(F)). Moreover, a transcriptional age index (TAI) analysis was performed, which is an indicator that quantitatively evaluates whether cells exhibit more gene expression characteristics of primitive unicellular organisms or of multicellular organisms. A higher TAI value indicates a stronger multicellular characteristic (a feature of normal cells), whereas a lower TAI value indicates a stronger unicellular characteristic (a feature of cancer cells). In general, cancer cells are known to have transcriptional immaturity and exhibit many expression characteristics of primitive unicellular organisms. As a result of the analysis, it was found that Cluster 1 exhibited greater transcriptional immaturity than Cluster 2 and differentiated cells. This means that the TAI value of Cluster 1, which is expected to have stronger cancer cell characteristics, was lower, indicating that features similar to primitive cells (a feature of cancer cells) were more prominently observed.

The signature scores of various cells associated with genes differentially regulated (DR) by ERCC3 were compared between Cluster 1 and differentiated cells. As a result, the expression of oncogenes among the genes regulated by ERCC3 was found to be higher in Cluster 1 (see FIG. 1(H)). A gene ontology (GO) analysis was performed on the gene set regulated by ERCC3. As a result, it was found that WNT signaling, cell cycle, and colorectal cancer-related signaling pathways were significantly enriched.

When the expression levels of the ERCC3 gene were compared and presented as a violin plot, the expression of ERCC3 in colorectal cancer organoids was found to be statistically significantly higher than in normal colon organoids (see FIG. 1(J)). Based on TCGA (The Cancer Genome Atlas) colorectal cancer patient data, a survival analysis according to ERCC3 expression levels was performed and presented as a Kaplan-Meier survival curve, and it was confirmed that the survival rate was significantly lower in the high ERCC3 expression group (see FIG. 1(K)).

### Mechanism of Cancer Reversion by ERCC3

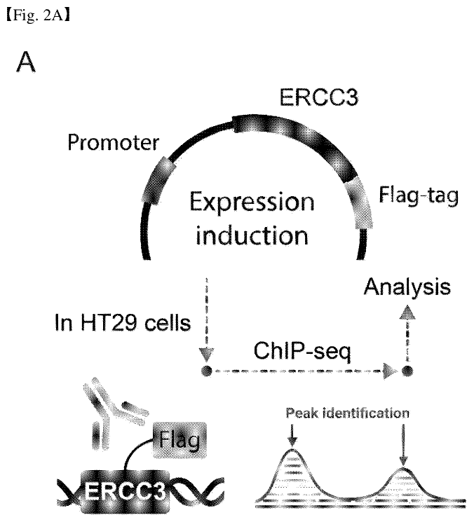

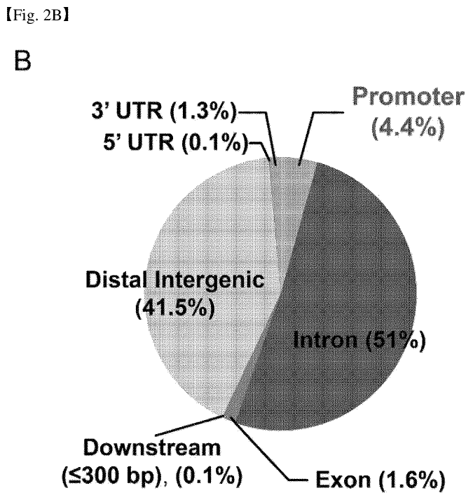

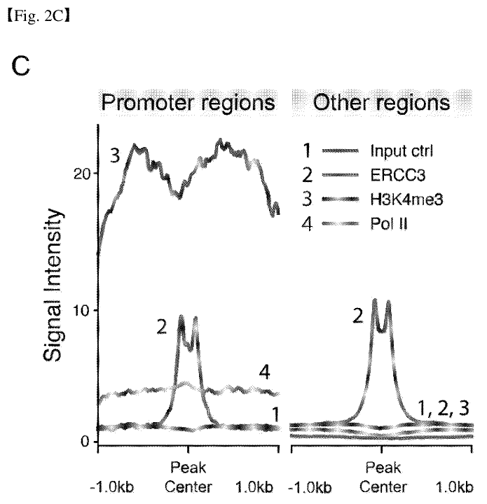

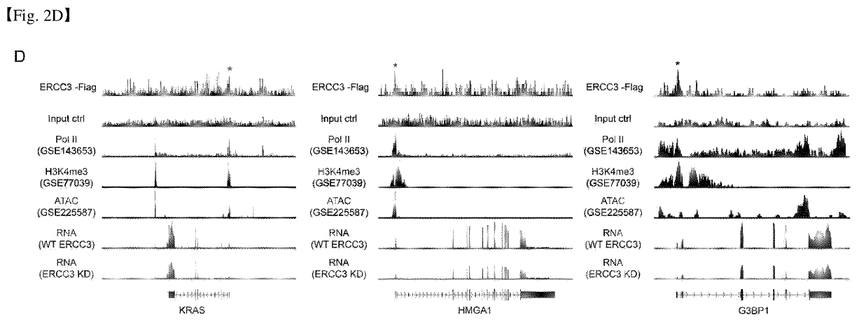

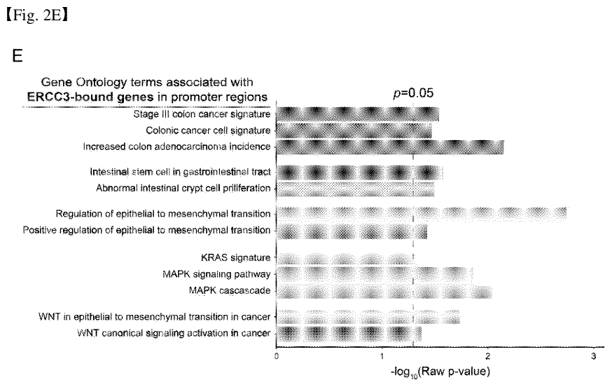

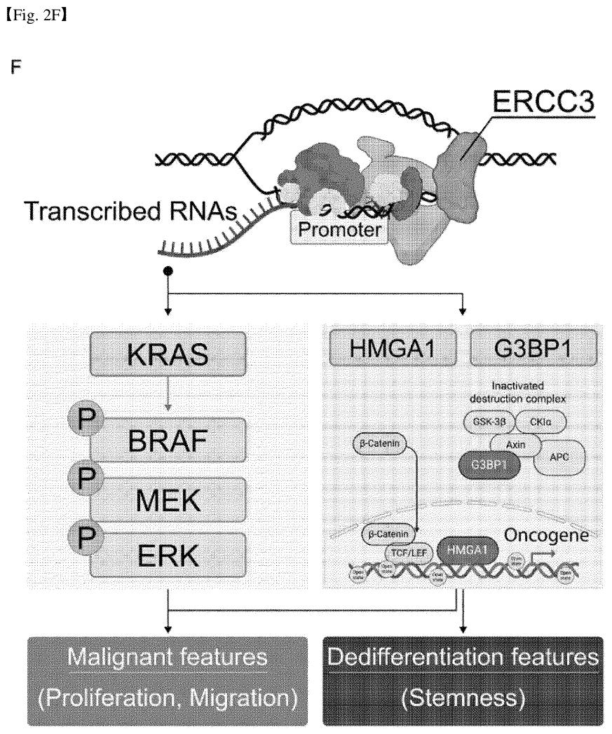

To specifically detect the binding sites of ERCC3, a FLAG-tagged ERCC3 gene was introduced into the colorectal cancer cell line HT29, and ChIP-seq experiments were performed to analyze the genome-wide DNA-binding sites of ERCC3. The experimental design of the ChIP-seq analysis to identify the DNA-binding sites of ERCC3 in the colorectal cancer cell line HT29 is shown in FIG. 1(A). As a result of the ChIP-seq analysis, it was found that more than 90% of the total binding sites of ERCC3 are located in distal intergenic and intron regions. This indicates that ERCC3 binds to sites involved in nucleotide excision repair (NER). Approximately 4.4% of the total binding sites are located in promoter regions of genes, in which case ERCC3 was found to play a role in regulating gene expression (see FIG. 2(B)). Transcription at the promoter regions requires the dissociation of hydrogen bonds in DNA, and reflecting this, an analysis was performed to determine whether signals of H3K4me3 (an active promoter marker) and RNA polymerase II were present around ERCC3 ChIP-seq peaks. As a result, it was found that signals of H3K4me3 (an active promoter marker) and RNA polymerase II were present together around ERCC3 peaks in the promoter regions (see FIG. 2(C)).

HMGA1 is an activator of the WNT signaling pathway and induces epigenetic modifications that facilitate the transcription of WNT target genes. G3BP1 (GTPase Activating Protein (SH3 Domain) Binding Protein 1) inhibits the function of the APC (Adenomatous Polyposis Coli) destruction complex, which mediates the degradation of β-catenin, thereby increasing the nuclear localization of β-catenin and promoting the transcription of WNT target genes. This complex is composed of APC, AXIN, GSK3B (Glycogen Synthase Kinase 3 beta), and CK1 (Casein Kinase 1), and induces ubiquitination and degradation of β-catenin through its phosphorylation. In addition, KRAS is a central gene in the RAS-MAPK signaling pathway and is involved in cell proliferation and survival. In particular, KRAS is known to drive the proliferation and progression of cancer cells through mutation or overexpression in various solid cancers, including colorectal cancer. The inventors have analyzed whether ERCC3 binds to the promoter regions of specific genes and, as a result, confirmed that ERCC3 specifically binds to the promoters of KRAS, HMGA1, and G3BPT. Inhibition of ERCC3 significantly reduced the expression of these genes, demonstrating that ERCC3 directly contributes to the transcriptional activity of these genes. KRAS plays a key role in the proliferation of cancer cells, whereas HMGA1 and G3BP1 are critical for the activation of the WNT signaling pathway and the maintenance of cancer cell sternness. This suggests that the regulation of ERCC3 can simultaneously inhibit major tumor-promoting pathways in cancer cells (see FIG. 2(D)).

A gene ontology (GO) analysis was performed to evaluate the common functions of the target genes of ERCC3. As a result, it was found that the genes bound by ERCC3 primarily contribute to the proliferation and development of colorectal cancer and, in particular, include features associated with malignant progression of colorectal cancer, such as epithelial-mesenchymal transition (EMT). It was confirmed that ERCC3 directly contributes to the activation of key oncogenic pathways by binding to the promoter regions of genes related to the KRAS and WNT signaling pathways (see FIG. 2(E)).

It was identified that ERCC3 promotes proliferation through KRAS expression and induces activation of the WNT signaling pathway, enhancement of sternness, and proliferation of cancer cells through HMGA1 and G3BP1 expression. This indicates that ERCC3 functions to enhance the cancer cell properties (proliferation and dedifferentiation features) of colorectal cancer (see FIG. 2(F)), and further suggests that inhibition of ERCC3 expression may serve as a critical molecular approach for the reversion of colorectal cancer cells.

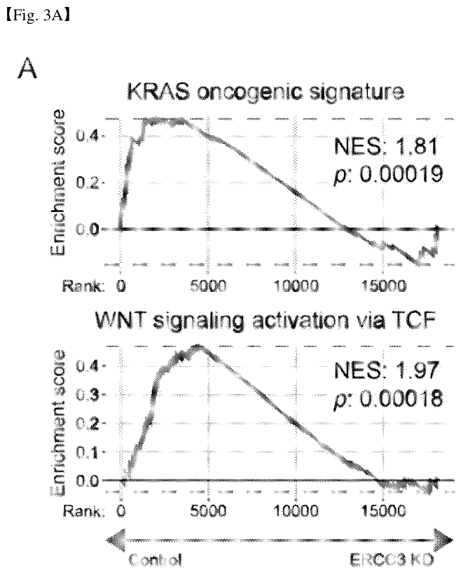

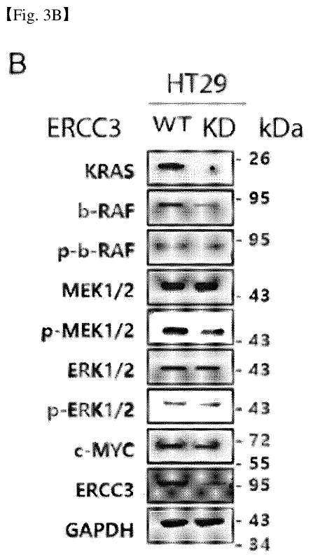

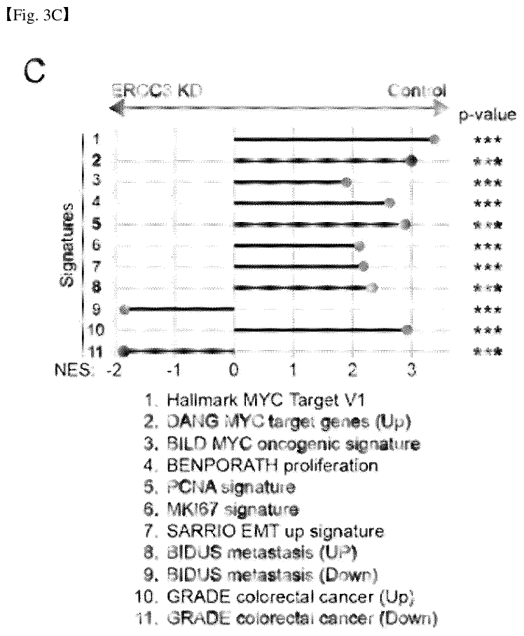

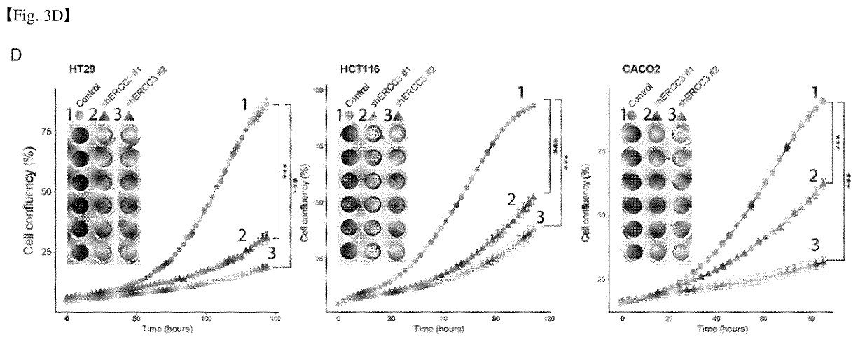

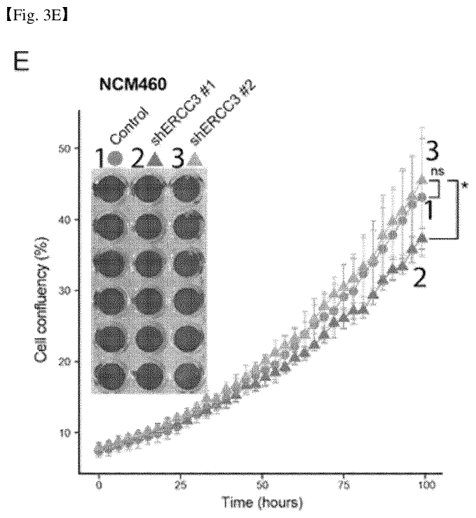

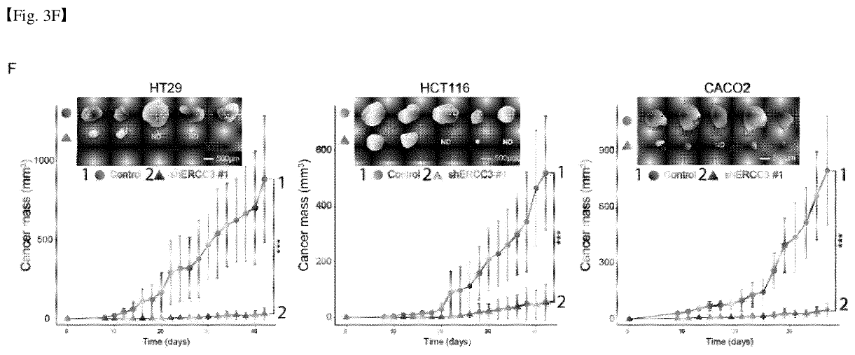

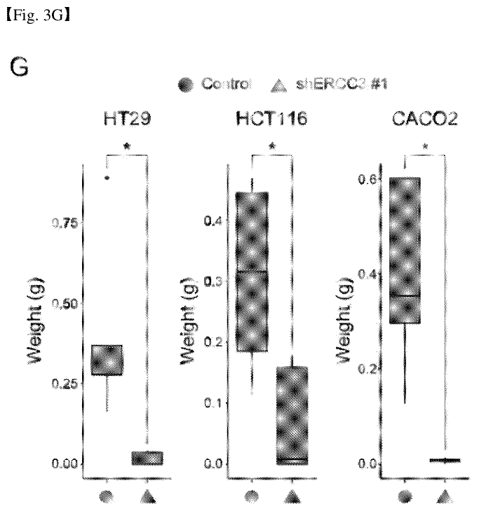

To verify that the unlimited proliferative capacity of colorectal cancer is closely associated with activation of the KRAS (associated with the MAPK pathway) and WNT signaling pathways, a gene set enrichment analysis (GSEA) was performed to evaluate whether a reduction in ERCC3 expression leads to suppression of these pathways. As a result, inhibition of ERCC3 was found to cause an overall decrease in the expression of genes related to the KRAS-MAPK and WNT signaling pathways (see FIG. 3(A)). This suggests that the reduction in ERCC3 expression can simultaneously inhibit the activation of the KRAS-MAPK and WNT pathways. Furthermore, to validate these results at the protein level, Western blot analysis was performed in the HT29 cell line. As a result, inhibition of ERCC3 was clearly found to reduce the activation of KRAS and its downstream MAPK signaling pathway (see FIG. 3(B)). Additionally, a gene ontology (GO) analysis was performed to evaluate how the activation of the KRAS and WNT signaling pathways actually affects downstream proliferation-related genes. As a result, it was found that the overexpression of colorectal cancer proliferation-related genes, including MYC, and key cell proliferation genes such as MK167 and PCNA, was regulated by ERCC3 (see FIG. 3(C)). This indicates that inhibition of ERCC3 suppresses the KRAS (MAPK pathway) and WNT signaling pathways, thereby effectively inhibiting the abnormal overexpression of cancer-related genes such as MYC and cell proliferation-related genes such as MK167 and PCNA in cancer cells.

### Therapeutic Effect of ERCC3 Inhibition on Cancer Reversion in Colorectal Cancer Cell Lines

The effect of ERCC3 inhibition on cell proliferation through cancer reversion was evaluated in three colorectal cancer cell lines (HT29, HCT116, and CACO2). In all three colorectal cancer cell lines, inhibition of ERCC3 using shERCC3 #1 of SEQ ID NO: 1 (GCCATTTCTAAGACTGCTGAA) and shERCC3 #2 of SEQ ID NO: 2 (CGGGAATATGTGGCAATCAAA) significantly suppressed the proliferation of colorectal cancer cells (see FIG. 3(D)). However, it can be predicted that ERCC3, a reversion target of colorectal cancer cells, has little or no effect on the proliferation of normal colon cells. Indeed, inhibition of ERCC3 in the normal colon epithelial cell line NCM460 resulted in little or no change in the cell proliferation rate (see FIG. 3(E)). This indicates that ERCC3 inhibition acts specifically on cancer cells and suggests the possibility of a therapeutic strategy without side effects.

### Therapeutic Effect of ERCC3 Inhibition on Cancer Reversion in Colorectal Cancer Xenograft Mouse Models

Three colorectal cancer cell lines (HT29, HCT116, and CACO2) were transplanted into mice to evaluate the effect of ERCC3 inhibition in vivo. The measurement of tumor volume over time showed that ERCC3 inhibition significantly reduced the tumor growth rate in all three colorectal cancer cell lines (see FIG. 3(F)). Moreover, the measurement of tumor tissue weight at the end of the experiment showed that the weight was significantly reduced in the ERCC3 inhibition group (see FIG. 3(G)).

### Verification of Reversion of Colorectal Cancer Through ERCC3 Inhibition

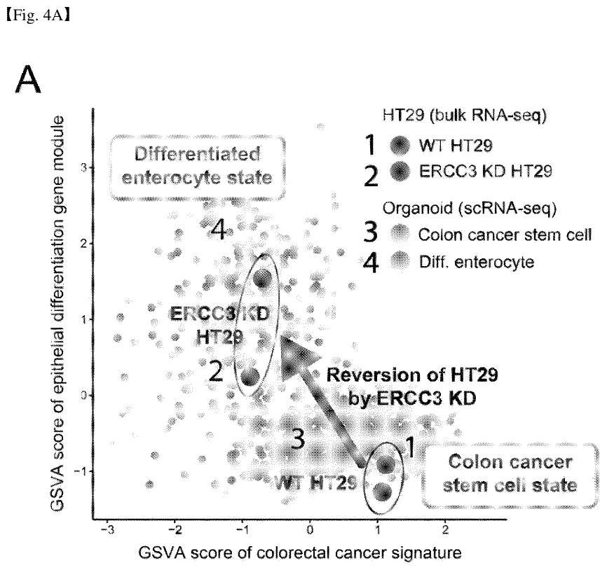

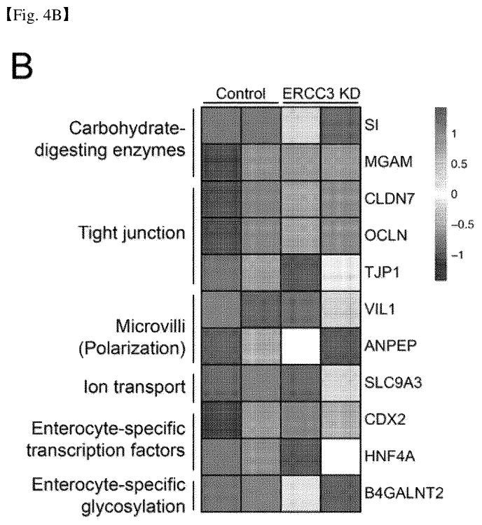

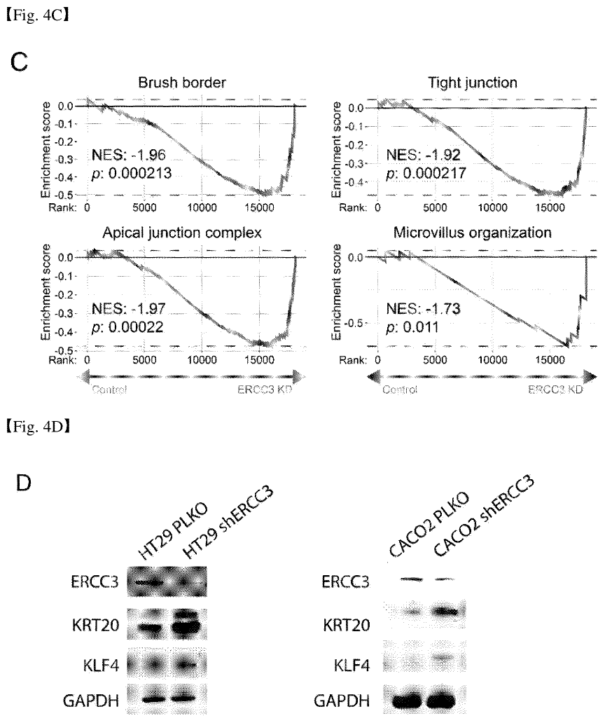

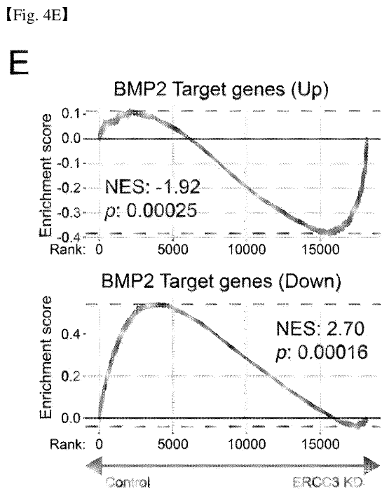

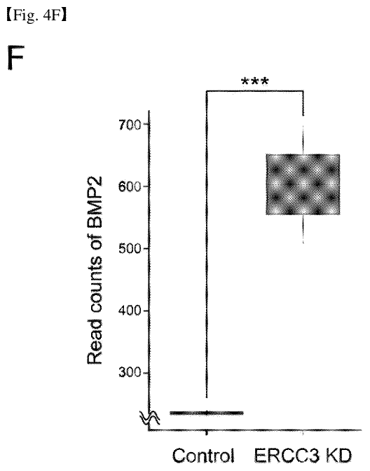

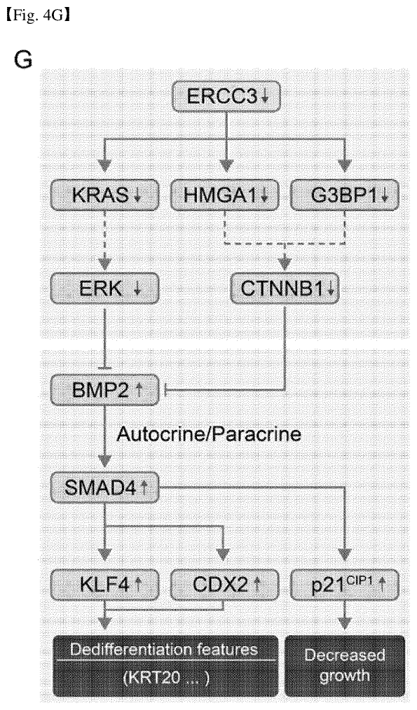

Inhibition of ERCC3 expression in the colorectal cancer cell line HT29 resulted in decreased cancer cell characteristics and increased differentiation-related traits of normal colon epithelial cells. Accordingly, it was confirmed that ERCC3 inhibition contributes to the induction of differentiation of colorectal cancer cells into normal cells. Furthermore, gene expression analysis was performed to evaluate whether the induction of normal differentiation in colorectal cancer cells leads to restoration of the expression of genes associated with normal colon functions. It was also confirmed that the expression of various genes associated with normal colon function, including colon-specific digestive enzyme secretion, tight junction formation, maintenance of microvilli structure, ion channels, and expression of colon-specific transcription factors, was increased upon inhibition of ERCC3. Upon inhibition of ERCC3, the expression of key genes essential for maintaining the polarity and barrier functions of colon epithelial cells, including genes related to the brush border, tight junction, and apical junction complex, which are representative structural features of normal colon tissue, increased (see FIGS. 4(A) to 4(C)).

The expression of KLF4 and KRT20, which are markers of normal enterocytes, was analyzed to verify the reversion of colorectal cancer. KLF4 was selected as a marker of normal colon epithelial cells because it is specifically expressed in these cells and acts as a transcription factor regulating the expression of intestinal alkaline phosphatase (IAP), which plays a crucial role in protecting the intestinal mucosa. KRT20 was also selected as a differentiation marker of colon epithelial cells, since it has been reported to be specifically expressed in differentiated enterocytes within normal colon tissue.

When ERCC3 was inhibited in two colorectal cancer cell lines (HT29 and CACO2), the expression of KRT20 and KLF4, which are differentiation markers of normal colon epithelial cells, was both statistically significantly increased (see FIG. 4(D)). This indicates that inhibition of ERCC3 expression induces differentiation of colorectal cancer cells.

This normal differentiation is closely associated with an increase in BMP2 expression. Normal colon epithelial cells begin to proliferate and undergo early differentiation in the crypt base, where WNT signaling is abundant. As the cells gradually migrate upward, they are exposed to an environment where WNT signaling decreases while BMP signaling increases, thereby leading to their transition into fully differentiated functional epithelial cells. In this process, BMP2 acts as a key signal that induces complete differentiation and functional restoration of the cells. ERCC3 was considered to play a role in maintaining such a differentiation-suppressive environment by itself. To verify this, gene set enrichment analysis (GSEA) and comparison of BMP2 expression levels demonstrated that inhibition of ERCC3 expression significantly increased BMP2 expression (see FIGS. 4(E) and 4(F)). As described above, inhibition of ERCC3 was found to suppress the activation of WNT signaling, and this suppression induced the activation of BMP2 signaling, thereby promoting normal differentiation of colorectal cancer cells. These findings provide mechanistic evidence that reduced expression of ERCC3 activates BMP2 signaling, leading to the normal differentiation of colorectal cancer cells into a state resembling normal enterocytes (see FIG. 4(G)).

### Reversion Therapy of Colorectal Cancer Using ERCC3-Selective Degrader Spironolactone

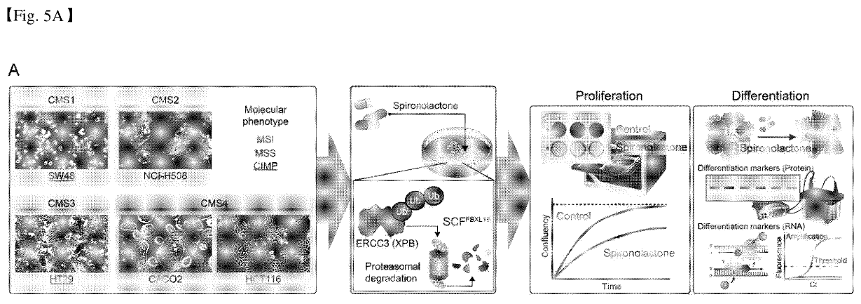

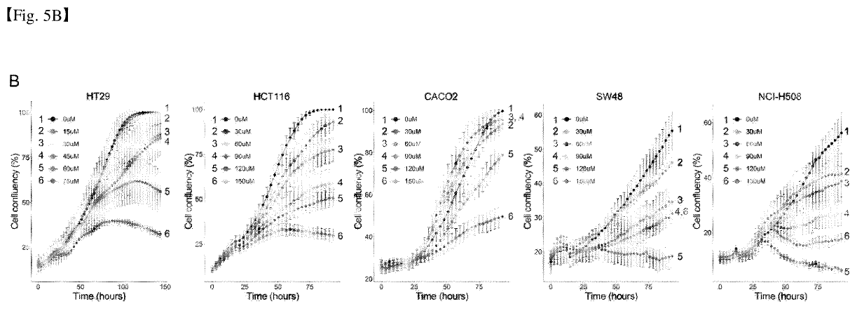

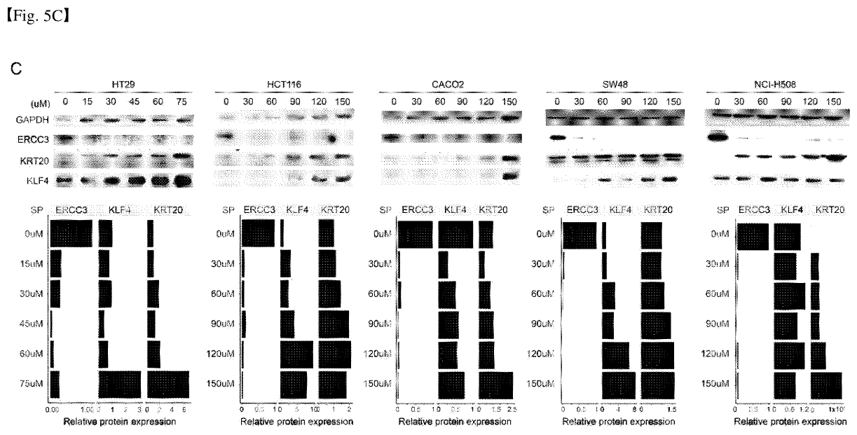

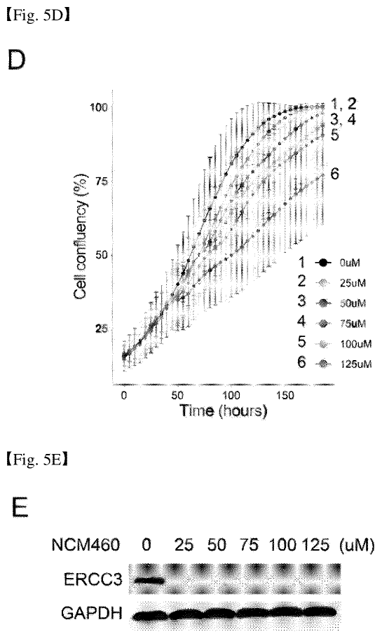

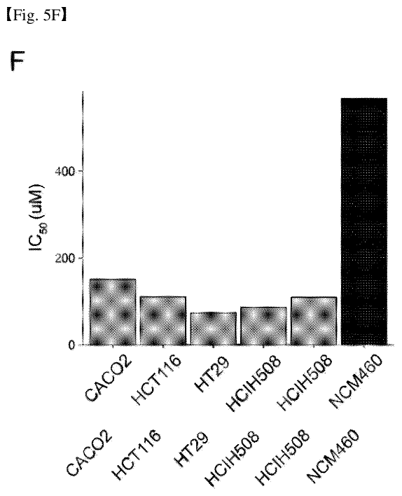

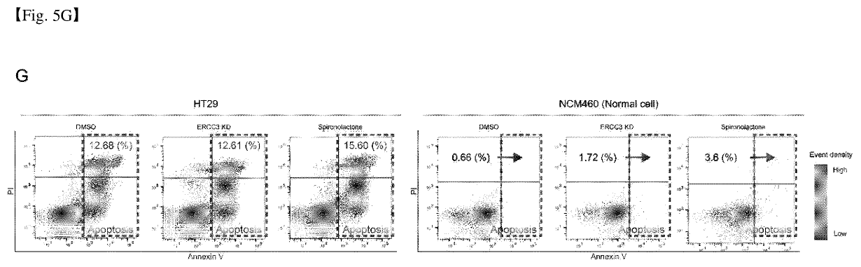

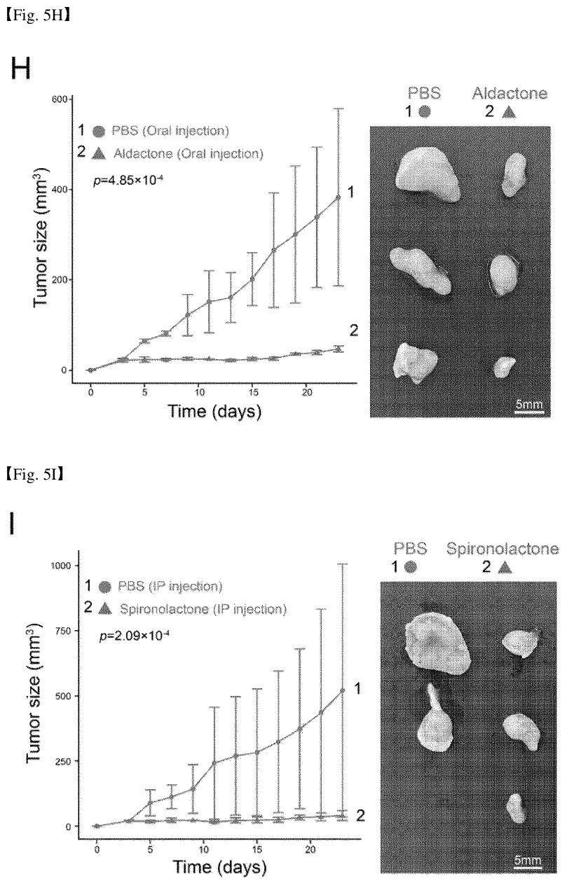

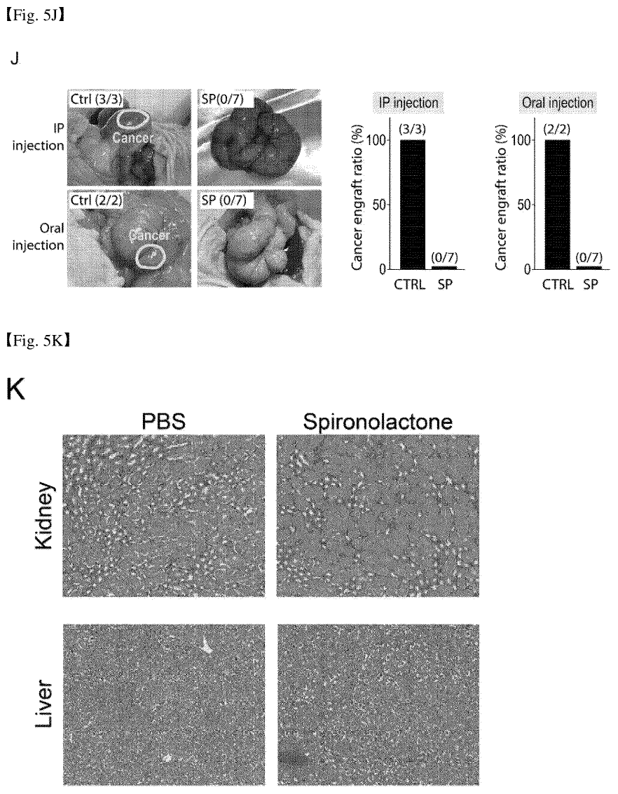

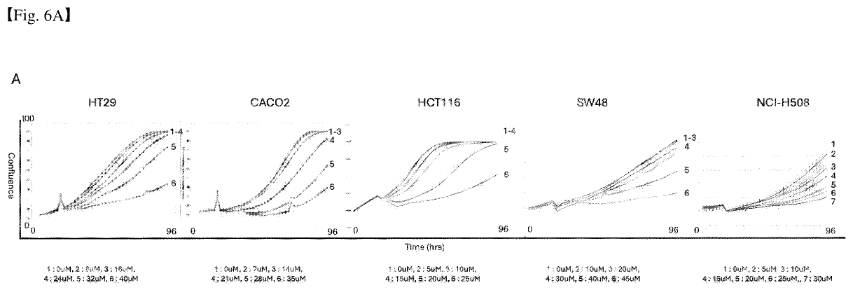

Spironolactone is a conventional drug known as an aldosterone receptor antagonist. However, it has recently been reported to reduce the protein level of ERCC3, a key component of the nucleotide excision repair (NER) pathway. Moreover, it has been shown to induce ubiquitination and proteasome-dependent degradation of ERCC3, thereby suppressing the activation of NER. Based on molecular characteristics, colorectal cancer is classified into four consensus molecular subtypes (CMSs): CMS1, CMS2, CMS3, and CMS4, and each CMS is known to exhibit distinct gene expression profiles and differences in drug responsiveness. Given that the expression of ERCC3 is crucial for maintaining cancer cell properties, it is hypothesized that a reversion therapy strategy targeting ERCC3 could be effective regardless of the CMS subtype. From this perspective, it was observed that the treatment of five colorectal cancer cell lines corresponding to consensus molecular subtypes (CMSs) 1-4 (CMS1: SW48, CMS2: NCI-H508, CMS3: HT29, and CMS4: HCT116 and CACO2) with spironolactone suppressed proliferation and induced differentiation through ERCC3 degradation (see FIG. 5(A)).

Moreover, the treatment of five colorectal cancer cell lines corresponding to consensus molecular subtypes (CMSs) 1-4 (CMS1: SW48, CMS2: NCI-H508, CMS3: HT29, and CMS4: HCT116 and CACO2) with spironolactone resulted in a dose-dependent decrease in cell proliferation and an increase in the expression of colorectal differentiation markers KRT20 and KLF4 (see FIGS. 5(B) and 5(C)). These results indicate that spironolactone induces ERCC3-specific degradation, thereby suppressing the proliferation of colorectal cancer cells and inducing their reversion into a state resembling normal-like cells (or normal-functioning cells). These findings also suggest that spironolactone may have relatively minimal effects on normal cells. In fact, when the normal colon cell line NCM460 was treated with spironolactone, little or no inhibition of cell growth was observed, unlike in colorectal cancer cell lines (see FIGS. 5(D) and 5(E)). Furthermore, when comparing the IC₅₀ values between the colorectal cancer cell lines and the normal colon cell line following spironolactone treatment, the IC₅₀ values of colorectal cancer cells were commonly below 200 μM, indicating their high sensitivity to spironolactone, whereas the normal colon cell line exhibited IC₅₀ values more than three times higher, demonstrating relatively greater resistance to cell proliferation inhibition (see FIG. 5(F)). Meanwhile, analysis of cell death in colorectal cancer cells following spironolactone treatment and ERCC3 inhibition revealed that inhibition or degradation of ERCC3 alone, either by genetic regulation or by drug treatment, induced little or no cell death (see FIG. 5(G)). Taken together, these findings suggest that spironolactone exerts a selective therapeutic effect in the reversion therapy of colorectal cancer by suppressing the proliferation of cancer cells while minimizing side effects on normal cells.

When Aldactone®, a spironolactone-based drug, was orally administered to mice implanted with colorectal cancer in the subcutaneous tissue and colon, both tumor size and proliferation rate were significantly reduced (see FIGS. 5(H) and 5(J)). Furthermore, when spironolactone was administered intraperitoneally to mice, the tumor-suppressive effect was reproduced, similar to that observed with oral administration (see FIGS. 5(I) and 5(J)). These findings demonstrate that spironolactone induces reversion of cancer cells by specifically degrading ERCC3, thereby suppressing the proliferation of colorectal cancer cells and simultaneously inducing normal differentiation, as evidenced both in vitro and in vivo. In addition, inhibition or degradation of ERCC3 exhibited excellent therapeutic efficacy against colorectal cancer, while no toxicity was observed in the liver or kidneys of mice (see FIG. 5(K)). These results suggest that, in future applications to colorectal cancer patients, the use of pharmaceutically acceptable salts based on ERCC3 inhibition or degradation of ERCC3 is expected to exhibit minimal side effects.

### Reversion Therapy of Colorectal Cancer Using ERCC3-Selective Inhibitor BPK-21

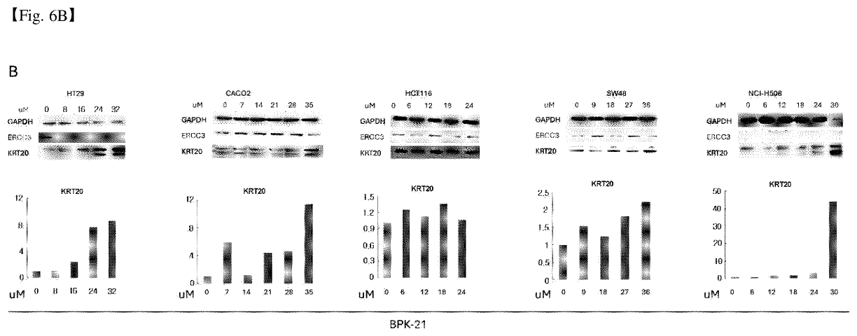

The treatment of five colorectal cancer cell lines (HT29, HCT116, CACO2, SW48, and NCI-H508) with BPK-21, a functional inhibitor (i.e., an enzyme activity inhibitor) of ERCC3, resulted in a significant decrease in cancer cell proliferation and a dose-dependent increase in the expression of the colorectal differentiation markers KRT20 and KLF (see FIGS. 6(A) and 6(B)). These findings suggest that BPK-21 effectively inhibits the function of ERCC3, thereby not only suppressing the proliferation of colorectal cancer cells but also inducing their transition (reversion) to a differentiated state similar to that of normal colon epithelial cells.

### Expansion of Cancer Reversion Therapy Using ERCC3-Selective Degrader Spironolactone

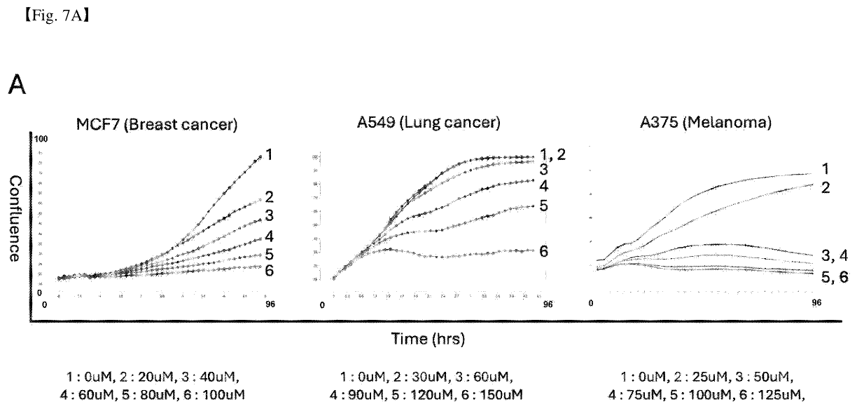

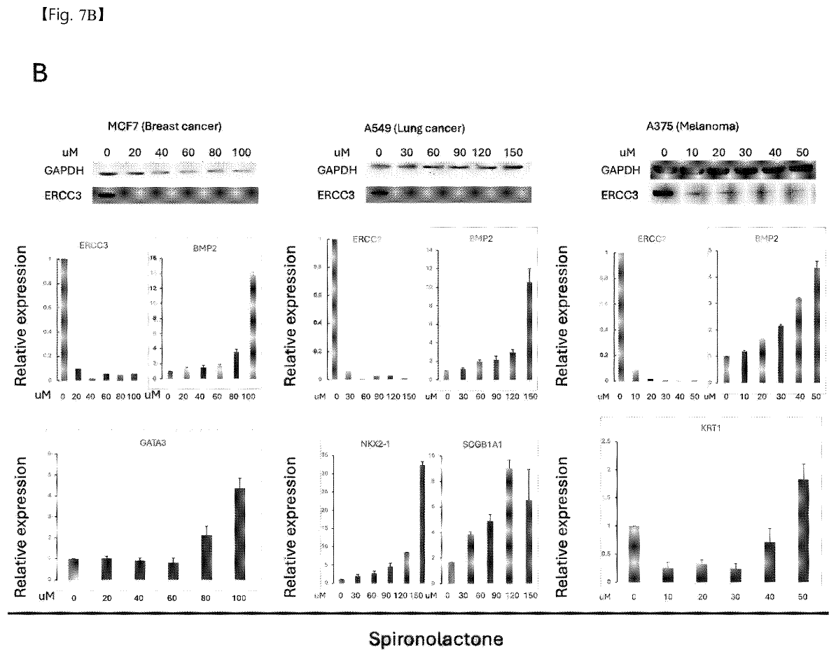

The treatment of the breast cancer cell line MCF7, the lung cancer cell line A549, and the skin cancer (melanoma) cell line A375 with spironolactone resulted in a dose-dependent decrease in cancer cell proliferation and a decrease in the expression of KRAS, which is involved in cancer cell proliferation. In addition, it was found that the expression of BMP2, a factor that induces cell differentiation, as well as the expression of cell differentiation markers, including: GATA3, a differentiation marker of normal breast luminal cells; NKX2-1 and SCGB1A1, differentiation markers of normal alveolar type I (AT1) lung cells; and KRT1, a marker of normal skin keratinocytes, were significantly increased (see FIGS. 7(A) and 7(B)). These findings demonstrate that spironolactone exerts a reversion therapy effect by degrading ERCC3, thereby inhibiting cancer cell properties and inducing differentiation even in cancer types (e.g., breast cancer, lung cancer, and skin cancer) other than colorectal cancer.

1. A pharmaceutical composition for preventing or treating cancer, comprising spironolactone represented by Chemical Formula 1 below or a pharmaceutically acceptable salt thereof, or BPK-21 represented by Chemical Formula 2 below or a pharmaceutically acceptable salt thereof, as an active ingredient,
wherein the composition reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation:

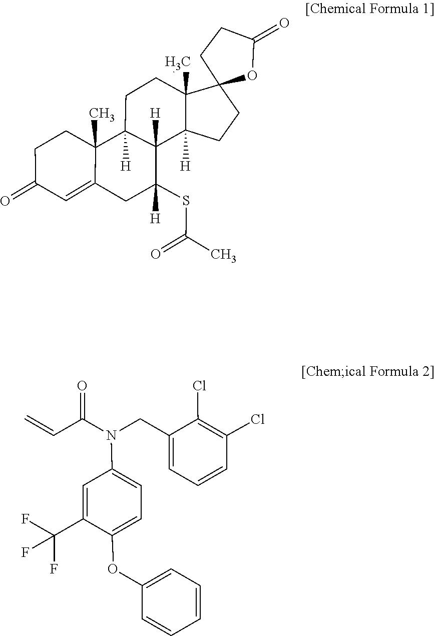

2. The pharmaceutical composition for preventing or treating cancer according to claim 1, wherein the composition reduces the expression of at least one gene selected from the group consisting of KRAS, HMGA1, and G3BP1.

3. The pharmaceutical composition for preventing or treating cancer according to claim 1, wherein the composition increases the expression of the BMP2 gene.

4. The pharmaceutical composition for preventing or treating cancer according to claim 1, wherein the composition reduces the overexpression of cancer-related genes such as MYC and cell proliferation-related genes such as MKI67 and PCNA in cancer cells.

5. The pharmaceutical composition for preventing or treating cancer according to claim 1, wherein the cancer is selected from the group consisting of colorectal cancer, breast cancer, lung cancer, and melanoma.

6. The pharmaceutical composition for preventing or treating cancer according to claim 5, wherein the cancer is colorectal cancer, and the composition increases the expression of KRT20, or KLF4, or both KRT20 and KLF4.

7. The pharmaceutical composition for preventing or treating cancer according to claim 5, wherein the cancer is breast cancer, and the composition increases the expression of GATA3.

8. The pharmaceutical composition for preventing or treating cancer according to claim 5, wherein the cancer is lung cancer, and the composition increases the expression of NKX2-1, or SCGB1A1, or both NKX2-1 and SCGB1A1.

9. The pharmaceutical composition for preventing or treating cancer according to claim 5, wherein the cancer is melanoma, and the composition increases the expression of KRT1.

10. A method for screening a drug that reverts cancer cells into normal-like cells (or normal-functioning cells) by specifically inhibiting or degrading ERCC3 (ERCC Excision Repair 3, TFIIH core complex helicase subunit), also referred to as XPB (Xeroderma Pigmentosum group B), thereby suppressing the proliferation of cancer cells and simultaneously inducing normal differentiation, the method comprising:
(a) treating cancer cells with a candidate substance in vitro;
(b) measuring the expression level of ERCC3 in the cancer cells treated with the candidate substance;
(c) selecting a candidate substance that exhibits a lower expression level of ERCC3 than that of a control group not treated with the candidate substance; and
(d) determining, among the candidate substances selected in step (c), a candidate substance that reduces the expression of at least one gene selected from the group consisting of KRAS, HMGA1, and G3BP1 and increases the expression of the BMP2 gene, as a drug that reverts cancer cells into normal-like cells (or normal-functioning cells).

11. The method for screening a drug that reverts cancer cells into normal-like cells (or normal-functioning cells) according to claim 10, wherein the drug that reverts cancer cells into normal-like cells (or normal-functioning cells) reduces the overexpression of cancer-related genes such as MYC and cell proliferation-related genes such as MKI67 and PCNA in cancer cells.

12. The method for screening a drug that reverts cancer cells into normal-like cells (or normal-functioning cells) according to claim 10, wherein the cancer is selected from the group consisting of colorectal cancer, breast cancer, lung cancer, and melanoma.

13. The method for screening a drug that reverts cancer cells into normal-like cells (or normal-functioning cells) according to claim 12, wherein the cancer is colorectal cancer, and the drug increases the expression of KRT20, or KLF4, or both KRT20 and KLF4.

14. The method for screening a drug that reverts cancer cells into normal-like cells (or normal-functioning cells) according to claim 12, wherein the cancer is breast cancer, and the drug increases the expression of GATA3.

15. The method for screening a drug that reverts cancer cells into normal-like cells (or normal-functioning cells) according to claim 12, wherein the cancer is lung cancer, and the drug increases the expression of NKX2-1, or SCGB1A1, or both NKX2-1 and SCGB1A1.

16. The method for screening a drug that reverts cancer cells into normal-like cells (or normal-functioning cells) according to claim 12, wherein the cancer is melanoma, and the drug increases the expression of KRT1.


SEQUENCE LISTING (2 sequences)

SEQ ID NO: 1 - RNA, 21 nt, synthetic construct
  source 1..21: mol_type = other RNA
gccatttctaagactgctgaa

SEQ ID NO: 2 - RNA, 21 nt, synthetic construct
  source 1..21: mol_type = other RNA
cgggaatatgtggcaatcaaa